(19) United States
(12) Patent Application Publication
(10) Pub. No.: US 2025/0349413 A1
(43) Pub. Date: Nov. 13, 2025

(54) MEDICAL VIDEO STREAMING WITH MACHINE LEARNING

(71) Applicant: Intuitive Surgical Operations, Inc., Sunnyvale, CA (US)

(72) Inventors: Moshe Bouhnik, Holon (IL); Daniel Dobkin, Tel Aviv (IL); Emmanuelle Muhlethaler, Tel Aviv-Yafo (IL); Roee Shibolet, Tel Aviv (IL)

(73) Assignee: Intuitive Surgical Operations, Inc., Sunnyvale, CA (US)

(21) Appl. No.: 19/203,827

(22) Filed: May 9, 2025

Related U.S. Application Data
(60) Provisional application No. 63/645,737, filed on May 10, 2024.

(51) Int. Cl. G16H 30/20; G16H 40/67; G16H 50/70

(52) U.S. Cl. CPC G16H 30/20; G16H 40/67; G16H 50/70

(57) ABSTRACT

Machine learning based efficient medical video streaming is described. A system can include one or more processors, coupled with memory, to receive, via a robotic medical system, a image frames related to a medical procedure performed by the robotic medical system. The one or more processors can transform, via one or more models trained with machine learning on historical images of medical procedures, the image frames to feature vectors. The one or more processors can cluster, via the one or more models, the feature vectors into clusters. The one or more processors can generate a run-length encoded data stream based at least in part on the clusters. The one or more processors can transmit, via a network, the run-length encoded data stream to one or more servers remote from the one or more processors to manage performance of the medical procedure.

### CROSS-REFERENCE TO RELATED PATENT APPLICATION

This application claims the benefit of priority to U.S. Provisional Patent Application No. 63/645,737, filed on May 10, 2024, which is hereby incorporated by reference herein in its entirety for all purposes.

### BACKGROUND

A robotic medical system can include an instrument for performing a medical session or procedure. For example, the instrument can be used to perform a surgery, a therapy, or a medical evaluation. The robotic medical system or a non-robotic medical system can collect videos or data of the medical procedure. For example, the robotic medical system can include an endoscope that collects the videos of the medical procedure. However, due to the large amounts of data collected by the robotic medical system, it can be challenging to stream the medical video data for real-time remote processing.

### SUMMARY

Technical solutions disclosed herein can include medical video streaming with machine learning. A computing system can efficiently stream a medical video to a remote server site while protecting private data in the medical video using machine learning. The computing system can use machine learning models that are trained on medical images to generate features, such as feature vectors, from the image frames of the medical video data. The computing system can implement machine learning models to generate clusters of feature vectors from a sequence of the feature vectors. In some implementations, the computing system can select a representative frame, such as a keyframe, from each cluster of feature vectors. The computing system can then encode the cluster and/or the selected key frame to generate an encoded data stream for transmission to a remote site. By using machine learning to compress the image into its features, the size of the video can be reduced, allow for less consumption of network bandwidth, a reduction in data storage, and allow for cloud processing while consuming less processor and memory resources. At the remote site, a computing system can decode the received data stream and re-create the medical video data. The decoding system can be customized, tuned, or otherwise tailored to the encoding system such that a generic decoding system would be unable to decode the data stream, thereby providing improved encryption of the data stream. For example, the computing system of the site can implement an encoder to encode the images into feature vectors, while the remote site can include a decoder trained with the encoder to decode the feature vectors back into the images. Further, by training the machine learning model using medical surgical data, this technical solution can improve the reconstruction of the medical images with high quality and using less data bits.

At least one aspect of the present disclosure is a system. The system can include one or more processors, coupled with memory, to receive, via a robotic medical system, image frames related to a medical procedure performed by the robotic medical system. The one or more processors can transform, via one or more models trained with machine learning on historical images of medical procedures, the image frames to feature vectors. The one or more processors can cluster, via the one or more models, the feature vectors into clusters. The one or more processors can generate a run-length encoded data stream via run-length encoding (or generate an encoded data stream with any other lossless data compression technique) based at least in part on the clusters. The one or more processors can transmit, via a network, the run-length encoded data stream to one or more servers remote from the one or more processors to manage performance of the medical procedure.

The one or more processors can train, using machine learning, the one or more models to reduce image loss between the image frames and the feature vectors.

The one or more processors can train, using machine learning, the one or more models to decrease entropy in the feature vectors.

The one or more processors can execute a function to train the one or more models that reduces image loss and decreases entropy in the feature vectors.

The one or more processors can receive a data set including the historical images. The one or more processors can filter the historical images to remove first images including private medical data from the data set and retain second images including non-private medical data. The one or more processors can train, using machine learning, the one or more models with the filtered historical images to generate first feature vectors for the first images, and generate second feature vectors for the second images, wherein images reconstructed from the second feature vectors have a level of accuracy that is less than images reconstructed from the first feature vectors.

One cluster of the clusters can include at least two of the feature vectors.

The one or more processors can train, using machine learning, an encoder to generate a feature vector from an image frame and a decoder to generate the image frame from the feature vector. The one or more processors can deploy the decoder to the one or more servers to execute on the one or more servers to transform the feature vector into the image frame responsive to the feature vector being extracted from the run-length encoded data stream.

The one or more processors can select a representative feature vector from feature vectors for a cluster of the clusters. The one or more processors can generate, using the representative feature vector, run-length encoded data to represent the feature vectors of the cluster.

The one or more processors can receive the run-length encoded data stream including run-length encoded data generated to represent feature vectors of a cluster. The one or more processors can generate, using the run-length encoded data stream, the feature vectors of the cluster. The one or more processors can decode, using one or more second models, the feature vectors of the cluster into at least a portion of the image frames.

The one or more processors can receive the run-length encoded data stream including run-length encoded data generated to represent feature vectors of a cluster. The one or more processors can generate, using the run-length encoded data, the feature vectors of the cluster. The one or more processors can classify, using one or more second models and the feature vectors of the cluster, each feature vector of the feature vectors of the cluster into a class of classes.

The one or more processors can receive the run-length encoded data stream including run-length encoded data generated to represent feature vectors of a cluster. The one or more processors can generate, using the run-length encoded data, the feature vectors of the cluster. The one or more processors can label, using one or more second models and the feature vectors of the cluster, an action performed by the robotic medical system represented in each feature vector of the feature vectors of the cluster.

The one or more processors can receive an indication of the medical procedure. The one or more processors can select, using the indication of the medical procedure, a bit rate. The one or more processors can quantize, using the bit rate, the clusters. The one or more processors can generate the run-length encoded data stream from the quantized clusters.

At least one aspect of the present disclosure is a method. The method can include receiving, by one or more processors, coupled with memory, via a robotic medical system, a image frames related to a medical procedure performed by the robotic medical system. The method can include transforming, by the one or more processors, via one or more models trained with machine learning on historical images of medical procedures, the image frames to feature vectors. The method can include clustering, by the one or more processors, via the one or more models, the feature vectors into clusters. The method can include generating, by the one or more processors, a run-length encoded data stream based at least in part on the clusters. The method can include transmitting, by the one or more processors, via a network, the run-length encoded data stream to one or more servers remote from the one or more processors to manage performance of the medical procedure.

The method can include training, by the one or more processors, using machine learning, the one or more models to reduce image loss between the image frames and the feature vectors.

The method can include executing, by the one or more processors, a function to train the one or more models that reduces image loss and decreases entropy in the feature vectors.

The method can include receiving, by the one or more processors, a data set including the historical images. The method can include filtering, by the one or more processors, the historical images to remove first images including private medical data from the data set and retain second images including non-private medical data. The method can include training, by the one or more processors, using machine learning, the one or more models with the filtered historical images to generate first feature vectors for the first images, and generate second feature vectors for the second images, wherein images reconstructed from the second feature vectors have a level of accuracy that is less than images reconstructed from the first feature vectors.

The method can include training, by the one or more processors, using machine learning, an encoder to generate a feature vector from an image frame and a decoder to generate the image frame from the feature vector. The method can include deploying, by the one or more processors, the decoder to the one or more servers to execute on the one or more servers to transform the feature vector into the image frame responsive to the feature vector being extracted from the run-length encoded data stream.

The method can include selecting a representative feature vector from feature vectors for a cluster of the clusters. The method can include generating, using the representative feature vector, run-length encoded data to represent the feature vectors of the cluster.

At least one aspect is directed to a non-transitory computer-readable medium storing processor-executable instructions that, when executed by one or more processors, cause the one or more processors to receive, via a robotic medical system, image frames related to a medical procedure performed by the robotic medical system. The instructions can cause the one or more processors to transform, via one or more models trained with machine learning on historical images of medical procedures, the image frames to feature vectors. The instructions can cause the one or more processors to cluster, via the one or more models, the feature vectors into a clusters. The instructions can cause the one or more processors to generate a run-length encoded data stream based at least in part on the clusters. The instructions can cause the one or more processors to transmit, via a network, the run-length encoded data stream to one or more servers remote from the one or more processors to manage performance of the medical procedure.

The instructions can cause the one or more processors to receive a data set including the historical images. The instructions can cause the one or more processors to filter the historical images to remove first images including private medical data from the data set and retain second images including non-private medical data. The instructions can cause the one or more processors to train, using machine learning, the one or more models with the filtered historical images to generate first feature vectors for the first images, and generate second feature vectors for the second images, wherein images reconstructed from the second feature vectors have a level of accuracy that is less than images reconstructed from the first feature vectors.

At least one aspect is directed to a non-transitory computer-readable medium storing processor-executable instructions that, when executed by one or more processors, cause the one or more processors to receive, via a robotic medical system, image frames related to a medical procedure performed by the robotic medical system. The instructions can cause the one or more processors to generate, via one or more models trained with machine learning on historical images of medical procedures, a plurality of clusters of a plurality of feature vectors based on the plurality of image frames. The instructions can cause the one or more processors to construct a run-length encoded data stream based at least in part on the plurality of clusters. The instructions can cause the one or more processors to transmit, via a network, the run-length encoded data stream to one or more servers remote from the one or more processors to manage performance of the medical procedure.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 depicts an example computing system to extract features of image frames of a medical procedure and generate a data stream from the features.

FIG. 2 depicts an example computing system to extract features from image frames, cluster the features, and select representative frames for the clusters.

FIG. 3 depicts an example computing system to generate a data stream from clustered features extracted from image frames.

FIG. 4 depicts an example computing system to generate clusters of features from a data stream.

FIG. 5 depicts an example computing system to perform operations on a cluster extracted from a data stream.

FIG. 6 depicts an example computing system to train an encoder to extract features from image frames and a decoder to reconstruct the image frames from the extracted features.

FIG. 7 depicts an example computing system to train a temporal encoder to generate a feature vector from a cluster of features and a temporal decoder to generate the cluster from the feature vector.

FIG. 8 depicts an example method of extracting features of image frames of a medical procedure and generating a data stream from the features.

FIG. 9 depicts an example method of training an encoder to extract features from image frames and a decoder to reconstruct the image frames from the extracted features.

FIG. 10 depicts an example computing architecture of a computing system.

### DETAILED DESCRIPTION

Following below are more detailed descriptions of various concepts related to, and implementations of, methods, apparatuses, and systems for medical video streaming with machine learning. The various concepts introduced above and discussed in greater detail below may be implemented in any of numerous ways.

This disclosure is generally directed to medical video streaming with machine learning. A medical facility, such as a hospital, outpatient center, or any other facility can include a robotic medical system that performs a medical procedure (e.g., surgery, therapy, or examination). The robotic medical system can include a camera, such as an endoscope, that captures videos, images, or frames during the medical procedure. The robotic medical system can transmit, send, push, stream, or deliver the frames to a computing system, computer, cloud platform, or server remote from the robotic medical system (e.g., an off-premises server outside the medical facility or a server in a different building or room of the medical facility). However, the amount of video data to be transmitted by the robotic medical system to the server can be large (e.g., megabytes of data, gigabytes of data, or terabytes of data). Transmitting a large amount of data to the server can utilize a large amount of bandwidth over a network connection. Therefore, there can be technical challenges in transmitting videos of medical procedures without consuming large amounts of network and computing resources. Some techniques such as using a video codec to compress a video, can be implemented. However, the video codec may not reduce the file size of the video enough to avoid consuming large amounts of network and computing recourse.

Because the videos are large, systems may not be able to transmit the videos in real-time. Streaming videos can consume a substantial portion of network bandwidth of a hospital. Hospital bandwidth can be even more adversely effected in situations where multiple procedures are performed simultaneously and a multiple videos are streamed simultaneously on the same hospital network. In this regard, the remote server may not be able to perform analysis on the video during the medical procedure itself. Recommendations, assistance, or insights that the remote server generates with the video may not be available to the operator of the robotic medical system until after the medical procedure is complete because the video may not be able to be delivered to the remote system for processing in real-time. Furthermore, the server system can utilize large amounts of cloud storage or cloud processing storage to store the videos received from the robotic medical system due to the large size of the videos.

Furthermore, videos captured by robotic medical systems can include private information, such as private identifying information (PII) or private health information (PHI). For example, the videos may include images of a face of a patient. Videos transmitted over a network connection may be accessed by an unauthorized third party. For example, techniques such as packet sniffing or network attacks can expose the videos, and the private information that the videos include, to the unauthorized third party.

To solve these, and other technical problems, technical solutions of this disclosure can include medical video streaming with machine learning. For example, systems and methods of this technical solution can include a video codec technique that is specific to compressing medical videos, and improve the efficiency with which medical video can be streamed in real-time to a remote server. A computing system of this technical solution can efficiently stream medical video data to a remote server site while protecting private data using machine learning. The computing system can implement machine learning to efficiently compress medical images, videos, or frames. The computing system can use machine learning models that are trained specifically on medical images to generate features, such as feature vectors, from the image frames of the medical video data. The computing system can implement machine learning models to generate a cluster from a sequence of the feature vectors. In some implementations, the computing system can select a representative frame, such as a keyframe, from each cluster of feature vectors. The computing system can then apply run-length encoding on the cluster and/or the selected key frame to generate an encoded data stream for transmission to a remote site. By using machine learning to compress the image into its features, the size of the video can be reduced, allow for less consumption of network bandwidth, lower network storage, and allow for cloud processing while consuming less processor and memory resources.

At the remote site, a computing system can decode the received run-length encoded data stream and re-create the medical video data. The decoding system can be customized, tuned, or otherwise tailored to the encoding system such that a generic decoding system would be unable to decode the data stream, thereby providing improved encryption of the data stream. For example, the computing system of the site can implement an encoder to encode the images into feature vectors, while the remote site can include a decoder trained with the encoder to decode the feature vectors back into the images. The encoder and decoder can be trained with medical data so that the encoder and decoder are specific to handling medical data.

Without the decoder trained with or based on the encoder, the remote computing system may be unable to decode features extracted from the run-length data stream back into the original image correctly. Using machine learning techniques, such as an encoder and a decoder architecture, can prevent a third party from assessing hacked or leaked video data features, while maintaining high video quality. Because the third party would need the specific decoder trained with the encoder that produced the feature vector of the images in order to decode the videos, the third party would be prevented from accessing the features and producing the image from the features.

The re-created medical video may not include any private information appearing in the original video. The computing system can avoid accurately representing or reproducing private data in images, and thus remove the private data from the videos. The encoder and decoder can be trained by machine learning to inaccurately decode or reproduce private information in the image from the feature vector. For example, the output image of the decoder may not accurately or precisely reproduce private information in the output image. In some implementations, the computing system can construct or generate a training data set of images by excluding or filtering out images or portions of images including private information. Because the encoder and decoder are not trained on private information, the encoder and decoder may be unable to convert an image into a feature vector and then back into the image without creating a blurry or lossy image that does not properly recreate the private information. For example, if the private information is the face of a patient, because the encoder and decoder are trained on a data set that excludes faces of patients, the decoder may not be able to clearly reproduce the face of a patient.

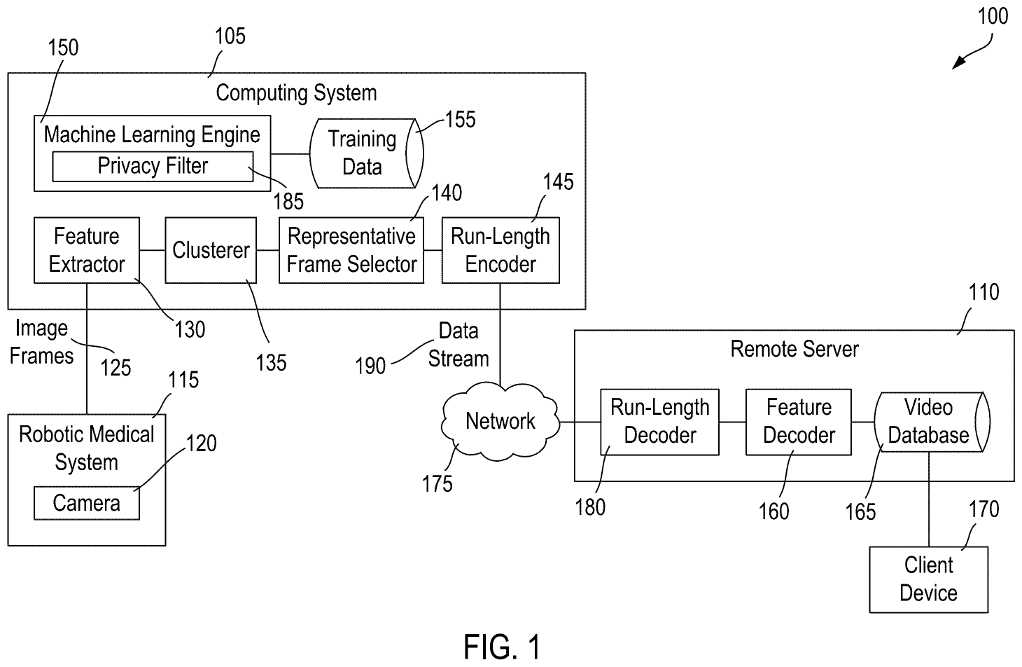

Referring now to FIG. 1, among others, a system 100 including an example computing system 105 to extract features of image frames 125 of a medical procedure and generate a data stream 190 from the features is shown. The system 105 can include at least one computing system 105. The computing system 105 can be a data processing system, a computer, a desktop computer, a control system, a console system, an embedded system, a cloud computing system, or any other type of computing system. The computing system 105 can be an on-premise computing system. The computing system 105 can be disposed on-premises within a medical facility. The medical facility can be a hospital, an outpatient center, or any other facility.

The system 100 can include at least one robotic medical system 115. The robotic medical system 115 can be a robotic system, apparatus, or assembly including at least one instrument. For example, the instrument can include an end or tip, such as a scalpel, a scissors, a monopolar curved scissors (MCS), a cautery hook tip, a cautery spatula tip, a needle driver, forceps, a round tooth retractor, a drill, or a clip applier. The instrument can be or include a robotic arm, a robotic appendage, a robotic snake, or any other motor controlled member that can be articulated by the robotic medical system. The instrument can include at least one actuator, such as a motor, servo, or other device. The instrument can be manipulated by motors, servo motors, actuators, or other devices to perform a medical procedure. The robotic medical system 115 can perform a medical session or medical procedure. For example, the robotic medical system 115 can articulate the instrument to perform surgery, therapy, or a medical evaluation with the instrument. A medical practitioner, such as a surgeon, technician, nurse, or other operator can provide input via a user device or input apparatus to manipulate the instrument to perform a medical procedure.

The robotic medical system 115 can be disposed on-premises within a medical facility. The medical facility can be a hospital, an outpatient center, or any other facility. The robotic medical system 115 can perform any type of medical procedure, such as a surgery, a therapy, or a medical evaluation. The robotic medical system 115 can be disposed at the same facility as the computing system 105. In some implementations, the robotic medical system 115 can be integrated with the computing system 105. For example, the computing system 105 can be a component of the robotic medical system 115.

The robotic medical system 115 can include at least one camera 120, in some implementations. The camera 120 can be or include an endoscope. For example, the camera can be an instrument that is manipulated by the medical practitioner and controlled via a motor, servo motor, or other input device of the robotic medical system 115. The robotic medical system 115 can produce image frames 125. The image frames 125 can be frames of a video captured by the camera 120 or images taken by the camera 120. The image frames 125 captured by the camera 120 of the robotic medical system 115 can track the medical procedure performed by the robotic medical system 115. The image frames 125 can capture instruments, anatomical structures (e.g., organs, muscles, bones, or skin), or the patient in the field of view of the camera 120. The robotic medical system 115 can send, transmit, provide, or push the image frames 125 to the computing system 105.

The computing system 105 can receive at least one image frame 125 from the robotic medical system 115. The image frames 125 received by the computing system 105 can be related to a medical procedure performed by the robotic medical system 115. For example, the image frames 125 can be a video or video stream of a medical procedure that the robotic medical system 115 performs. During the medical procedure the robotic medial system 115 can provide the video to the computing system 105. In some implementations, after the medical procedure is complete, the robotic medical system 115 can provide the image frames 125 to the computing system 105.

In some implementations, the system 100 may not include the robotic medical system 115. For example, the computing system 105 can receive image frames 125 from a non-robotic medical system, such as an endoscopic or video recording system. For example, the image frames 125 can be non-robotic videos, such as colonoscope or endoscope videos. Furthermore, the system 100 can be applied to execute on other types of data modalities. For example, the data 125 can be depth images received from a depth imaging system, computed tomography (CT) scans received from a CT system, ultrasound data received from an ultrasound system, magnetic resonance imaging (MRI) data received from an MRI system, etc.

The computing system 105 can implement or execute a pipeline of multiple steps, operations, machine learning blocks, machine learning models, or machine learning functions to encode the image frames 125 into a data stream 190 and transmit the data stream 190 to a remote server 110. The pipeline can include at least one feature extractor 130, at least one clusterer 135, at least one representative frame selector 140, and at least one run-length encoder 145. Each operation of the pipeline can be performed sequentially so that a video of the image frames 125 flows through the computing system 105 and is delivered in an encoded form to the remote server 110. In some implementations, the operations or phases of the pipeline are different models, for example, the pipeline can be a machine learning pipeline. For example, the feature extractor 130 can be a first model to generate a feature vector of an image, the clusterer 135 can be a second model to cluster feature vectors of image frames 125, and the representative frame selector 140 can be a third model to identify or select a representative frame, such as a keyframe, for each cluster. In some implementations, the clusterer 135 can generate clusters of various sizes. For example, the clusterer 135 can generate a cluster of a first size and a second cluster of a second size different than the first size. In some implementations, the size of the cluster can be based on the amount of motion of the instruments of the robotic medical system 115. The more motion of the instruments, the smaller the size of the clusters. By selecting the size of the cluster based on the amount of motion of the instruments, for example, the computing system 105 can dynamically adjust the cluster size to balance the quality of the data stream with the size of the data stream. The computing system 105 can include computing resources to execute the pipeline in real-time as the image frames 125 are received.

The feature extractor 130 can be or include at least one model. The feature extractor 130 can transform the image frames 125 into at least one feature or set of features. For example, the feature extractor 130 can embed an image frame 125 into a set of features, such as a feature vector. The feature extractor 130 can transform multiple image frames 125 each into a distinct feature vector. The feature extractor 130 can produce multiple different feature vector, each for a distinct image frame 125. For example, the feature extractor 130 can transform a first image frame 125 into a first feature vector and transform a second image frame 125 into a second feature vector. The feature extractor 130 can encode kinematics information or other data (e.g., event stream) into the feature vectors.

The feature extractor 130 can be or include at least one model trained by machine learning, such as a deep neural network, to extract feature vectors from the image frames 125. The feature extractor 130 can be an encoder, such as an encoder from an encoder-decoder neural network topology or architecture. The feature extractor 130 can be a vision or image transformer, in some implementations. The feature extractor 130 can be a neural network that is trained by a machine learning technique to produce a hidden internal state or feature state representation of the image frames 125 that can be used by the feature decoder 160 to transform the features back into the images 125.

For example, the computing system 105 can include at least one machine learning engine 150. The machine learning engine 150 can train the feature extractor 130 and the feature decoder 160. The feature extractor 130 can be trained by the machine learning engine 150 based on all or a portion of the training data 155. The feature extractor 130 and the feature decoder 160 can be models trained based on self-supervised machine learning by the machine learning engine 150. The self-supervised machine learning technique can include joint embeddings, e.g., self-distillation with no labels (DINO) or masked Siamese network (MSN), auto encoders (AE), decoder-encoder models, masked auto-encoders (MAEs). The machine learning engine 150 can train the feature extractor 130 and the feature decoder 160 can be trained with a supervised learning method.

The computing system 105 can provide the feature vectors extracted by the feature extractor 130 to the clusterer 135. For example, the computing system 105 can transition or move sequences of feature vectors through the pipeline, e.g., from the feature extractor 130 to the clusterer 135. The feature extractor 130 or the computing system 105 can cause the feature vector to be time-ordered, for example, the feature vectors can be ordered to correspond to the order of the frames in the video captured by the robotic medical system 115. The feature vectors can each be tagged or linked to a different time stamp to represent the order of frames 125 in the video.

The clusterer 135 can be another machine learning based block, module, function or model. The clusterer 135 can cluster similar frames together. For example, the clusterer 135 can cluster the feature vectors together. The clusterer 135 can wait for a predefined number of feature vectors to be received from the feature extractor 130, and execute clustering responsive to the predefined number of feature vectors being received from the feature extractor 130. The clusterer 135 can cluster, via one or more models, the feature vectors produced by the feature extractor 130 into one or multiple different clusters. Each cluster can include at least two feature vectors. In some implementations, a cluster can include only one feature vector.

The clusterer 135 can be a model or algorithm trained by machine learning to output a group or an indication of a group or cluster of multiple feature vectors. The clusterer 135 can implement a variety of different clustering techniques, methods, or processes, such as centroid-based clustering, density-based clustering, or distribution-based clustering. The clusterer 135 can perform temporal action segmentation. The clusterer 135 can detect segments of action and inaction, and segment the medical procedure video into clusters representing different actions of various lengths and with start and end times. The clusterer 135 can implement temporal action detection for medical procedure videos with a sparse set of actions. The clusterer 135 can implement temporally-weighted hierarchical clustering for unsupervised action segmentation (TWFINCH).

Once the image frames 125 are clustered, the clustered feature vectors can be provided by the clusterer 135 to the representative frame selector 140. Each cluster of feature vectors can be provided to the representative frame selector 140. The representative frame selector 140 can select an optimal, representative, or keyframe feature vector of the cluster. The representative frame selector 140 can select a feature vector to act as the representative frame from the cluster. The representative frame selector 140 can select an optimal representation of the cluster that helps reduce data size for the encoded data stream to be transmitted to the remote server 110 but allows for high restoration quality by the remote server 110 converting the encoded data stream back into images. Such a representation can be a keyframe feature vector with minimal or low difference of dissimilarity to all other frames in the cluster.

The representative frame selector 140 can select at least one keyframe for each cluster. The representative frame selector 140 can select one keyframe per cluster. The representative frame selector 140 can select multiple keyframes per cluster. For example, the representative frame selector 140 can determine a number of keyframes to select for a cluster based on the size of the cluster. The representative frame selector 140 can use a medoid selection process to select the keyframe for a cluster. Each keyframe can be a medoid for a cluster. For example, the representative frame selector 140 can perform medoid selection by selecting a keyframe by identifying a frame that has a minimal dissimilarity to all other feature vectors of the cluster. The keyframe can be a mean or centroid, in some implementations. The representative frame selector 140 can generate a link or relationship between the keyframe feature vector for each cluster and the other feature vectors of the cluster. For example the keyframe can be marked or otherwise identified with a flag.

The computing system 105 can provide the clustered features to the run-length encoder 145. The computing system 105 can provide the representative feature vector for each cluster to the run-length encoder 145. In some implementations, the computing system 105 can perform temporal encoding to encode differences between feature vectors of particular clusters, quantize the resulting temporally encoded feature vectors, and implement a scan operation before providing the data to the run-length encoder 145.

The run-length encoder 145 can generate a data stream 190. The data stream 190 can be a run-length encoded data stream or run-length code. The data stream 190 can include a stream or set of packets, messages, pieces of information, data, or binary information that represents encoded or compressed information (e.g., encoded versions of the feature vectors of the image frames 125). That run-length code can be transmitted to a destination for the video of the medical procedure, e.g., the remote server 110. The run-length encoder 145 can generate the run-length encoded data stream 190 using the clustered feature vectors. The run-length encoder 145 can generate the run-length encoded data stream 190 using both the clustered feature vectors and the representative feature vector. The run-length encoded data stream 190 can represent the clusters of feature vectors. The run-length encoded data stream 190 can represent the representative feature vectors. In this regard, a representation of each cluster of feature vectors can be encoded via an run-length code algorithm. The run-length encoder 145 can implement a run-length encoding (RLE) algorithm or technique to replace sequential or consecutive data elements as a single value or count of that data element. For example, the symbol sequence “AAABBC” could be compressed by RLE to the symbol sequence “3A2BC”. In some implementations, the encoder 145 can implement RLE. In some implementations, instead of RLE, the encoder 145 can implement any other lossless data encoding or compression technique. For example, the encoder 145 can implement a lossless data compression technique such as entropy coding or encoding. The lossless data compression technique can be, CABAC, CAVLC, or any other entropy encoding technique. In some implementations, the encoder 145 can implement a lossy compression technique, such as a discrete cosine transform (DCT) based compression algorithm (e.g., H.261, Motion JPEG, MPEG, etc.).

The computing system 105 can transmit, send, push, or communicate the data stream 190 to the remote server 110. The computing system 105 can transmit the data stream 190 over, via, or using at least one network 175. The network 175 can communicably couple the computing system 105 with the remote server 110. The network 175 can be or include a local area network (LAN) within a medical facility. The network 175 can include a Wi-Fi network. The network 175 can include a wired Ethernet network. The network 175 can include an Internet connection or the Internet. The computing system 105 and the remote server 110 can implement Internet Protocol (IP) (e.g., IPv4, IPV6), transmission control protocol (TCP), or tag distribution protocol (TDP) protocols to communicate.

The system 100 can include at least one remote server 110. The remote server 110 can be disposed remote from the computing system 105. The remote server 110 can be disposed in a different physical building, location, state, or country from the medical facility where the robotic medical system 115 and the computing system 105 are located. In some implementations, the remote server 110 is located in the same medical facility as the robotic medical system 115 or the computing system 105 but in a different building, different room, or different wing of the medical facility. The remote server 110 can generate data for the robotic medical system 115 to use in performing the medical procedure. For example, the computing system 105 can transmit, via the network 175, the run-length encoded data stream 190 to the remote server 110 to manage performance of the medical procedure. For example, the remote server 110 can perform various operations, such as classifying anatomical structures, segmenting anatomical structures, detecting actions or events of the medical procedure, generating objective performance indications, generating suggestions for the operator of the robotic medical system 115, generating control setting updates for controlling an endoscope or instrument by the robotic medical system 115, etc. The remote server 110 can operate using the received data stream 190 and send data back to the robotic medical system 115, such as metrics, suggestions, control commands, or control setting updates to operate the robotic medical system 115.

The system 100 can include at least one remote server 110. The remote server 110 can be or include a computing system, computer, cloud platform, or server remote from the robotic medical system (e.g., an off-premises server outside the medical facility). The remote server 110 can include at least one run-length decoder 180, at least one feature decoder 160, and at least one video database 165. The remote server 110 can implement or execute a pipeline of multiple steps, operations, machine learning blocks, machine learning models, or machine learning functions to decode the received data stream 190. The pipeline can include at least the run-length decoder 180, the feature decoder 160, and the video database 165. Each operation of the pipeline can be performed sequentially so that the data stream 190 flows through the pipeline and is decoded. In some implementations, the operations or phases of the pipeline are different models, for example, the pipeline can be a machine learning pipeline. The remote server 110 can decode data in real-time, and can include a level of processing resources to execute the pipeline in real-time.

The run-length decoder 180 can be a decoder that transforms the data stream 190. The run-length decoder 180, alone or with other components, can transform the data stream 190 into clusters of feature vectors, at least one vector that encodes differences between feature vectors of a cluster, or a representative feature vector of a cluster. For example, the computing system 105 can generate, using the run-length encoded data stream 190, feature vectors of clusters encoded in the data stream 190. The run-length decoder 180 can be implement decoding of an RLE algorithm or technique to transform a single value or count of a data element back into an original representation of the data, e.g., transform the symbol sequence the symbol sequence “3A2BC” back to the symbol sequence “AAABBC.” The decoder 180 can implement decoding for any lossless data compression technique such as H.264, H.265, or AV1. The run-length decoder 180 can implement decoding for a lossy compression technique, such as a discrete cosine transform (DCT) based compression algorithm (e.g., H.261, Motion JPEG, MPEG, etc.).

The feature decoder 160 can decode, using one or more second models, feature vectors of the cluster into at least a portion of the image frames 125. The feature decoder 160 can decode clusters of feature vectors back into image frames 125. For example, the feature decoder 160 can transform a feature vector of a cluster back into the image frame 125. The feature decoder 160 can be a model, such as a neural network model. The feature decoder 160 can be a model trained with the feature extractor 130, and form an encoder-decoder model architecture. For example, the feature decoder 160 can be a model that receives one feature vector, or a set of feature vectors for a cluster, and outputs a reconstructed image frame corresponding to the one feature vector, or a set of reconstructed image frames corresponding to the set of feature vectors for the cluster.

The remote server 110 can include at least one video database 165. The video database 165 can store images reconstructed from features vectors of the feature decoder 160. The database 165 can be or include a vector database. The database 165 can be or include a structured query language (SQL) database, a not only SQL (noSQL) database, a Redis database, a library, or any other data repository or storage system. The remote server 110 can store a set, collection, or group of videos, images, or video clips in the database 165. The remote server 110 can store the embeddings of the videos, images, or video clips video in database 165. The remote server 110 can store the clusters of the videos, images, or video clips in the video database 165.

The video database 165 can store feature vectors extracted from the data stream 190. In some implementations, the video database 165 can store feature vectors instead of videos. This can result in less storage resources of the video database 165 being consumed because the feature vectors can be a compressed representation of the video. In this regard, the video database 165 can store more videos while consuming less database storage. When a client device 170 requests to view a particular video, the feature decoder 160 can retrieve the feature vectors corresponding to the video from the video database 165, transform the feature vectors into the image frames of the video, and provide the reconstructed video to the client device 170. The client device 170 can be a laptop, desktop computer, a console, a smartphone, a tablet, or any other mobile or stationary computing system.

The computing system 105 can include at least one machine learning engine 150. The machine learning engine 150 can be implemented by the computing system 105, the remote server 110, the robotic medical system 115, or any other type of computing device. The machine learning engine 150 can implement machine learning techniques, training techniques, or learning techniques to train the models of the computing system 105 or the remote server 110. For example, the machine learning engine 150 can implement various supervised or self-supervised training algorithms to train, tune, or configure the parameters, values, or weights of the models of the computing system 105 or the remote server 110.

For example, the machine learning engine 150 can train the feature extractor 130 and the feature decoder 160 together in an encoder-decoder model architecture. The machine learning engine 150 can train the feature extractor 130 and the run-length decoder 180 with training data 155. The training data 155 can be historical image frames of medical procedures. The training data 155 may only include historical image frames of medical procedures, and not other types of images, e.g., images of cars or vehicles, images of movies or shows, images outside a patients body. Because the training data 155 can be image frames of medical procedures, and not other types of image frames, the resulting feature extractor 130 and feature decoder 160 can be specific to encoding and decoding image frames of internal procedures on a patient. In this regard, the resulting trained feature extractor 130 and feature decoder 160 can be specific to medical procedures, and thus result in a more efficient compression of image frames 125 compared to other more general image encoding or compression techniques. For example, the machine learning models trained specifically on medical data can compress the image frames 125 to smaller data sizes than general image encoding or compression techniques.

The image encoding of the feature extractor 130 and the feature vector decoding of the feature decoder 160 can be customized and specific to medical surgical images. Similarly, the clusterer 135 can be trained to cluster specific medical frames. The resulting cluster based encoding using machine learning can be customized and specific to medical surgical images. In some implementations, the models are trained with image frames of different medical procedures and models is produced for different types of medical procedures. In this regard, the models can be customized for different types of medical procedures. The computing system 105 can identify the type of medical procedure that the image frames 125 are captured for, and utilize the corresponding feature extractor 130 to encode the image frames 125. Likewise, the remote server 110 can identify the particular type of medical procedure, and implement a feature decoder 160 trained specifically for that type of medical procedure to decode the feature vectors. By customizing the models for specific types of medical procedures, for example, aspects of this technical solution can improve the encoding and decoding of the medical video data stream using less data bits, while maintaining the image quality. For example, the computing system 105 can select machine learning models to perform the encoding based on the type of medical procedure captured by the video stream. When streaming the encoded data stream to the remote server 110, the computing system 105 can provide an indication of the type of medical procedure, or other indication of the decoding technique to use to reliably and accurately reconstruct the video stream.

The machine learning engine 150 can include at least one privacy filter 185. The computing system 105 or the privacy filter 185 can receive a data set of historical images, e.g., the training data 155. The privacy filter 185 can be a filter that removes images from the training data 155 that include private information, PII, or PHI. The privacy filter 185 can be a filter designed to recognize private information in images. For example, the privacy filter 185 can be a model trained by machine learning to detect images that include private information. For example, the model can be a neural network, a classifier, an encoder-decoder model, or a convolutional neural network that can identify whether an image input into the model includes private information. For example, the privacy filter 185 can exclude images (or portions of images) that are out of patient images or include the face of a patient. The privacy filter 185 can retain images that do not include private information. For example, endoscope images taken by the camera 120 within the patient can be retained by the privacy filter 185. In some implementations, the privacy filter 185 can distinguish between image frames 125 that are in-body images, images captured within a patient, and out-of-body images, images taken outside the body of the patient. The privacy filter 185 can filter out out-of-body images and retain in-body-images for training the models (e.g., as the in-body-images are non-private images). This can result in high quality restoration of in-body images, but poor restoration of out-of-body images, thus protecting private information, e.g., the face of a patient or other person.

The privacy filter 185 can filter the historical images of the training data 155 to remove first images that include private medical data and retain second images including medical data that is not private or second images that do not include any private information (e.g., is non-private). Non-private information/images can include in-body images, and not out-of-body images. The non-private information/images may not include any private information, PII, or PHI. The privacy filter 185 can update the training data 155 to remove or discard any image including private data or exclude that image from training. For example, the privacy filter 185 can delete or discard an image from the training data 155, or apply a label, tag, or other identifier to the image so that the image is excluded from training.

The machine learning engine 150 can train the various models of the computing system 105 and the remote server 110 with the updated training data 155. For example, the machine learning engine 150 can use machine learning techniques, processes, or algorithms to train one or more models with the filtered historical images of the training data 155. The models can be trained to generate first feature vectors for images that do not include private data with a first level of accuracy, but generate second feature vectors for second images that do include private data with a second level of accuracy, less than the first level. Because the training data does not include images including private information, an encoder-decoder model that transforms an image with private information into a feature vector, and then reconstructs the image will be inaccurate because the training data 155 does not include any image representative of private information. In this regard, the encoding of images including private information into feature vectors, and back into reconstructed version of the images can have a lower accuracy than images that do not include private information because the encoding and decoding is trained on training data 155 that does not include any private data. The resulting reconstructed images that include private information can be blurred or blurry, or be poor or inaccurate reconstructions so that the private information in the reconstructed image is not visible or is blurry.

Because the training data 155 does not include images with private information, the models can be trained passively to blur images. However, the machine learning engine 150 can actively train models to blur images or portions of images. For example, the privacy filter 185 can identify private information in historical images, and apply a blur to the private information or remove or block out the private information. The privacy filter 185 can update the training data 155 with the updated images. The machine learning engine 150 can train the models with the updated images, and because the portion of the images of the training data 155 are blurry, fuzzed out, or excluded the models can be actively trained to blur or hide private information. This active or passive privacy training can result in security or privacy features built directly into the models that the machine learning engine 150 produces.

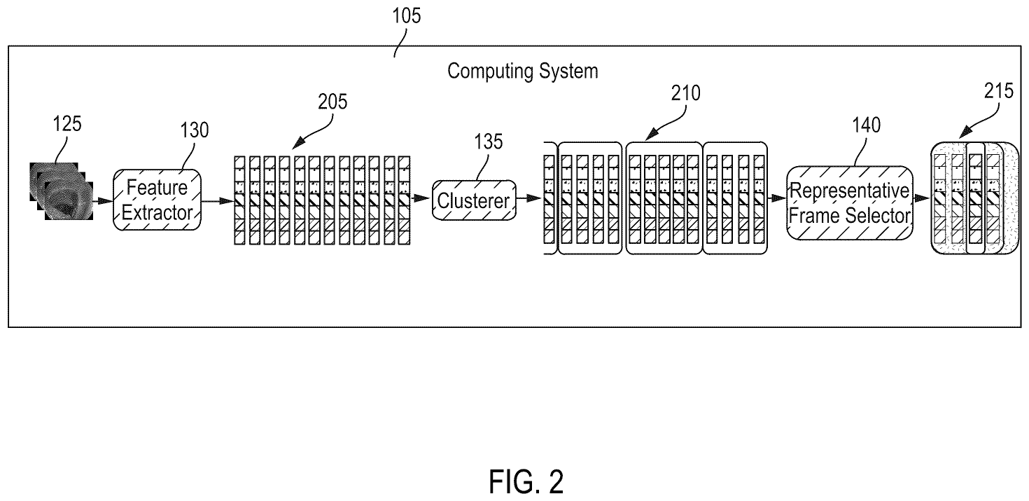

Referring now to FIG. 2, among others, an example computing system 105 to extract features from image frames, cluster the features, and select representative frames for the clusters is shown. The feature extractor 130 can receive the image frames 125 as an input and generate feature vectors 205. The image frames 125 can be time-ordered frames of a longer video, and can be processed one by one through the feature extractor 130 to produce a sequence of feature vectors 205. Each feature vector 205 can correspond to a different image frame 125. The feature vectors 205 can be ordered in a series by the computing system 105 to represent the order of the image frames 125. Each feature vector 205 can be a hidden internal state or feature state representation of the image frames 125 output by an encoder of the feature extractor 130. Each feature vector 205 can be a vector of values, numbers, or data entries for a variety of dimensions or data elements that represent a corresponding image frame 125.

The clusterer 135 can produce the clusters 210 from the feature vectors 205. Each cluster 210 can include one or more feature vectors 205. The feature vectors 205 can be clustered temporally, such that the feature vectors 205 can remain in order after clustering and within each cluster. The feature vectors 205 can be clustered based on similarities. For example, each cluster 210 can include similar feature vectors 205. Each cluster 210 can represent a different segment of the video of the medical procedure. For example, each cluster 210 can include a starting time and an ending time. Feature vectors 205 associated with timestamps between the start time and the end time can correspond to the cluster 210.

The representative frame selector 140 can select a representative frame 215 for each cluster 210. The representative frame selector 140 can identify, tag, or flag a particular feature vector 205 of each cluster 210 to represent the clusters 210. The representative frame selector 140 can select a representative feature vector 215 by identify the representative feature vector 215 as a medoid or centroid of a cluster 210. For example, the representative feature vector 215 can be a medoid of a cluster 210 that has a minimal dissimilarity to each other feature vector 205 in the cluster 210.

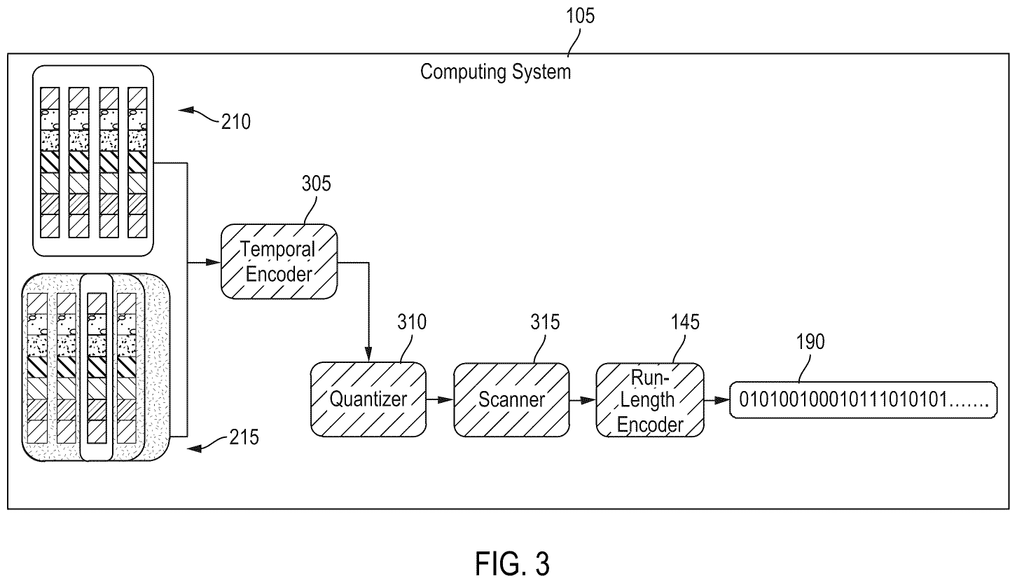

Referring now to FIG. 3, among others, an example computing system 105 to generate a data stream 190 from clustered features 205 extracted from image frames 125 is shown. The computing system 105 can include at least one temporal encoder 305. The temporal encoder 305 can be an encoder model, such as an encoder of an encoder-decoder neural network. The temporal encoder 305 can receive the cluster 210 as an input. The temporal encoder 305 can receive the representative feature vector 215 as an input. In some implementations, the temporal encoder 305 receives the cluster 210 but not the representative feature vector 215. The temporal encoder 305 can encode differences or changes between the feature vectors 205 of a particular cluster 210. The temporal encoder 305 can output a feature vector representing the feature vectors of the cluster 210 and encoding the differences between the feature vectors 205 of the cluster 210. The temporal encoder 305 can generate the encoding of the cluster 210 using the feature vectors 205 of the cluster 210.

The computing system 105 can include at least one quantizer 310. The quantizer 310 can perform quantization using one or a series of feature vectors output by the temporal encoder 305 for a series of clusters 210. The quantizer 310 can map the feature vectors output by the temporal encoder 305 from a first larger set to a second smaller set to reduce the size of the series of feature vectors output by the temporal encoder 305. The quantizer 310 can map from the first larger set to the second smaller set with a bit rate setting. The quantizer 310 can adjust or set the bit rate for quantization based on a variety of different data inputs.

For example, the computing system 105 can receive an indication of a type of the medical procedure that the video being compressed is taken of. The quantizer 310 can include a table or relationship indicating that for a particular medical procedure type, a specific bit rate should be used by the quantizer 310. Responsive to selecting the bit rate, the quantizer 310 can quantize, using the selected bit rate, the clusters 210. The resulting run-length encoded data stream 190 can be generated from the quantized clusters 210. In some implementations, the bit rate is selected to be a facility wide bit rate. For example, different hospitals can have different bit rates, and the bit rate selected and implemented by the quantizer 310 can be based on the hospital where the video was recorded. Furthermore, the bit rate can be selected based on a priority level for a medical procedure, or a desired quality of service. A high priority level medical procedure can be quantized with a high bit rate, while a low priority level medical procedure can be quantized with a lower bit rate. The computing system 105 can select the bit rate for a particular medical procedure data stream based on what other medical procedure data streams at a particular medical site (e.g., hospital) are being transmitted simultaneously or in an overlapping manner. In some implementations, the bit rate for the quantizer 310 is set based on surgeon or operator of the robotic medical system 115. Different surgeons can be associated with different profiles that can indicate to transmit data streams using different bit rates.

The quantized feature vectors can be provided to a scanner 315. The computing system 105 can include at least one scanner 315. The scanner 315 can execute a scan operation, technique, or algorithm. The scanner 315 can organize the quantized data based on whether symbols or values of the quantized data repeat. For example, the scanner 315 can organize blocks of data such that more repeating symbols appear near one end of a larger data frame. The run-length encoder 145 can generate the data stream 190 from the output of the scanner 315.

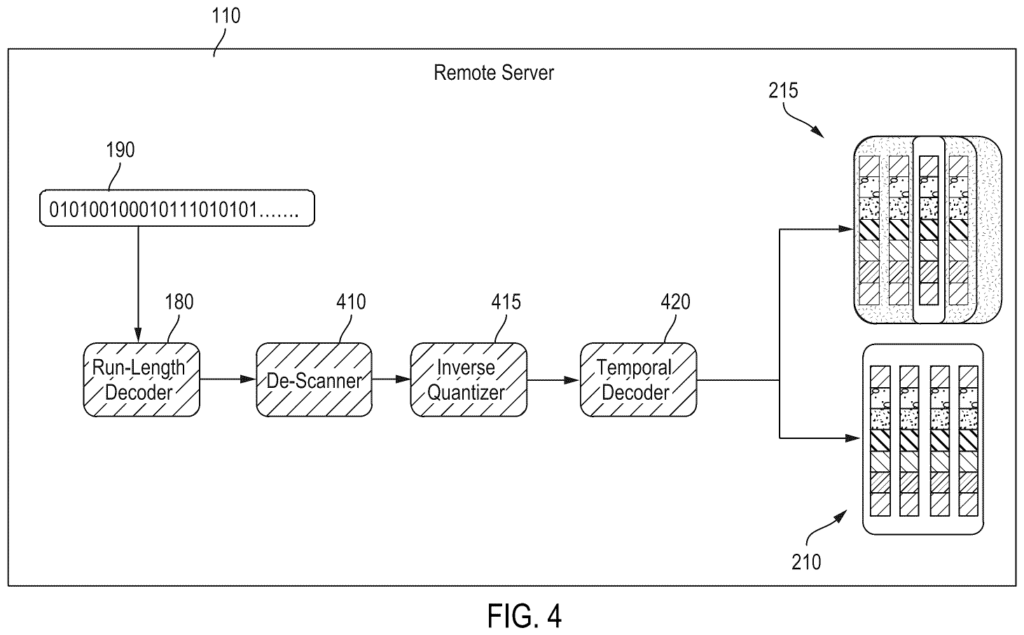

Referring now to FIG. 4, among others, an example computing system 110 to generate clusters of features from a data stream is shown. The remote server 110 can receive the data stream 190 from the computing system 105. The remote server 110 can be or can implement decoding infrastructure, e.g., a decoding station that is remote from a physical location (e.g., operating room) where the robotic medical system 115 and the computing system 105 are located. The remote server 110 can implement a machine learning pipeline of at least one machine learning model. The machine learning models of the remote server 110 can match the models of the computing system 105, or can be trained with the models of the computing system 105. For example, if the model of the remote server 110 is a decoder, that decoder can be trained with a corresponding encoder implemented on the computing system 105. In some implementations, the operations of the remote server 110 can be inverse operations of the operations of the computing system 105 and the remote server 110 can implement the inverse operations in the reverse order that the computing system 105 performed the operations.

For example, the data stream 190 can be decoded by a run-length decoder 180 that decompresses or decodes the run-length encoded data stream 190 produced by the run-length encoder 145. The remote server 110 can implement a de-scanner 410 that reverses or undoes the scanning performed by the scanner 315. For example, the de-scanner 410 can put data blocks back into their original order. The de-scanned data can be provided to an inverse quantizer 415. The inverse quantizer 415 can transform the lower sized dataset back into the larger dataset using the quantize rate. For example, the inverse quantizer 415 can reconstruct the original feature vectors output by the temporal encoder 305. The inverse quantizer 415 may reproduce a version of the feature vector output by the temporal encoder 305, for example, the reconstruction can include loss or error introduced through the quantizer 310 and the inverse quantizer 415. The output of the inverse quantizer 415 can be provided to a temporal decoder 420. The output can be a reconstruction of the feature vector that encodes differences between feature vectors of a cluster 210.

The temporal decoder 420 can transform the feature vector into the cluster 210 of feature vector 205. The temporal decoder 420 can transform the feature vector into the representative feature vector 215 of the cluster 210. The temporal decoder 420 can be a decoder of an encoder-decoder model including the temporal encoder 305. The temporal decoder 420 can be trained with the temporal decoder 305 to transform a feature vector produced by the temporal encoder 305 into the cluster 210 of feature vectors 205 or representative feature vector 215. If the temporal encoder 305 is trained to encode the cluster 210 of feature vectors 205, the temporal decoder 420 can decode or reconstruct the cluster 210 of feature vectors 205. If the temporal encoder 305 is trained to encode the cluster 210 with the representative feature vector 215, the temporal decoder 420 can reproduce both the cluster 210 of feature vectors 205 and the representative feature vector 215.

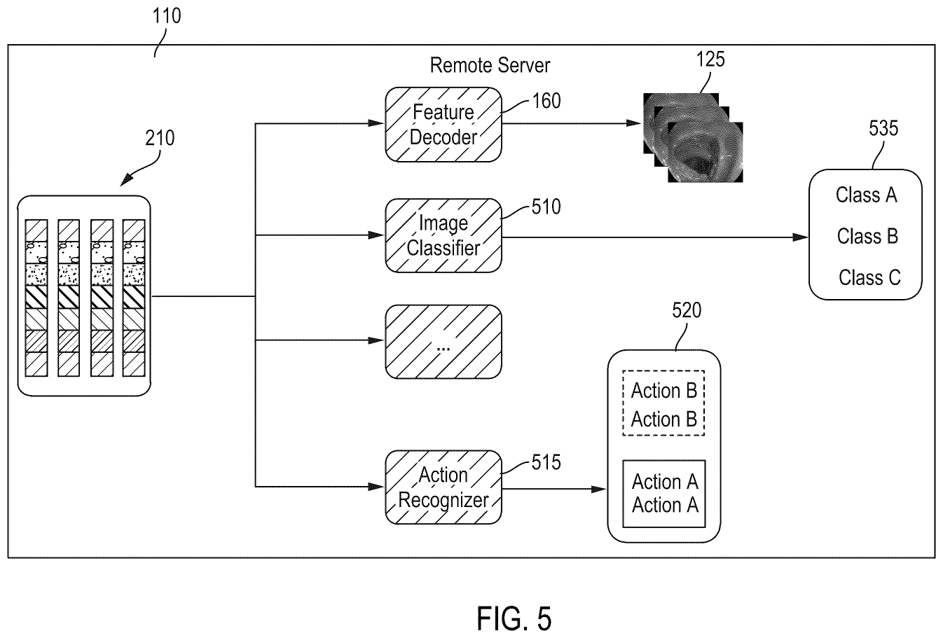

Referring now to FIG. 5, among others, an example computing system 110 to perform operations on a cluster 210 extracted from a data stream 190 is shown. The remote server 110 can implement a variety of different operations, models, or functions that operate directly on the cluster 210 of feature vectors 205 or directly on feature vectors 205. For example, instead of reconstructing the image frames 125 from the feature vectors 205 and executing the models on the image frames 125, the models can be executed directly on the feature vectors 205 representing the image frames 125. This can reduce the consumption of processor resources and memory resources to re-encode the images 125. Furthermore, this can reduce storage resource consumption because the remote server 110 may store the clusters 210 of feature vectors 205, which may be smaller in size than the image frames 125 that the clusters 210 and feature vectors 205 represent.

The remote server 110 can implement the feature decoder 160 to reproduce or reconstruct the image frames 125 from the cluster 210 of feature vectors 205. Furthermore, the remote server 110 can include an image classifier 510. The image classifier 510 can be a neural network that executes on the cluster 210 of feature vectors 205 to classify information in each image frame 125 or classify each image frame 125. The image classifier 510 can classify each feature vector 205 into a class 535 of a set or group of available classes 535. The image classifier 510 can output a class 535. For example, the image classifier 510 can output a medical procedure type for each feature vector 205, an identification of an anatomical structure represented in the feature vector 205, an identification of an instrument represented in the feature vector 205, or an identification of a state of an anatomical structure in the feature vector.

The remote server 110 can implement an action recognizer 515. The action recognizer 515 can label each feature vector 205 with an indication of an action depicted within the feature vector 205. For example, the action recognizer 515 can label individual feature vectors 205 with an indication of an action in the feature vectors 205, or can label an entire cluster 210 of feature vectors 205 as depicting a particular action. The action recognizer 515 can output an action 520 for each feature vector 205 or each cluster 210 of feature vectors 205. The action can be an action performed by the robotic medical system 115, for example, cutting, creating an incision, opening an anatomical structure, reconstructing an anatomical structure, sewing up an incision, or cauterizing an area of an anatomical structure. The action recognizer 515 can output an action 520 for a set of actions or group of actions that the action recognizer 515 is trained to recognize.

In some implementations, the remote server 110 can generate performance indicators or metrics from the feature vectors 205 or the clusters 210 of feature vectors 205. The remote server 110 can generate performance indicators based on a performance of encoding of the feature vectors 205. For example, the remote server 110 can determine or measure a level of entropy in a feature vector 205, in a sequence of feature vectors 205, or in a cluster 210 of feature vectors 205. The level of entropy in the feature vectors 205 can indicate a performance level (e.g., efficiency level or skill level) of the operator of the robotic medical system 115 to perform a medical procedure. The level of entropy in the feature vectors 205 can indicate an amount of motion of an instrument used to perform the medical procedure. For example, the greater the entropy, the lower the performance of the operator of the robotic medical system 115. In some implementations, the entropy based performance metric can be generated on-premises by the computing system 105 instead of, or in addition to, the remote server 110. The computing system 105 can transmit the entropy based performance metrics to the remote server 110 for analytics purposes or to construct a dashboard graphical user interface.

In some implementations, the feature decoder 160, the image classifier 510, or the action recognizer 515 can be downstream tasks that train directly on the cluster 210 of feature vectors 205 instead of on images. Having downstream models train directly using the feature vectors 205 produced by the feature extractor 130 can save processing and storage costs, because it can removes the need to decode the feature vectors 205 into the images 125 as part of training the downstream models.

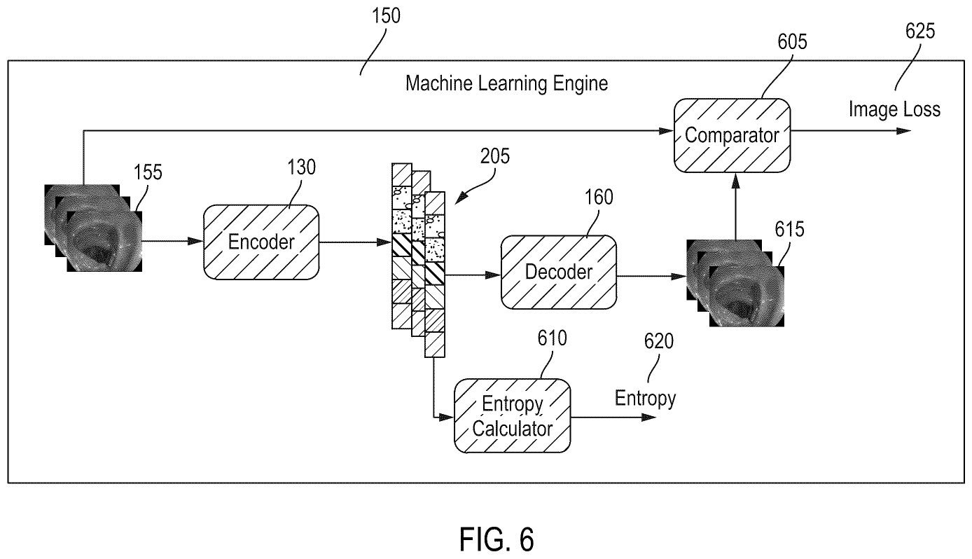

Referring now to FIG. 6, among others, an example computing system 105 to train an encoder 130 to extract features 205 from image frames 155 (e.g., training data) and a decoder 160 to reconstruct the image frames 155 from the extracted features 205. The computing system 105 can implement a machine learning engine 150 to train the encoder 130 and the decoder 160. The encoder 130 can be trained to extract the feature vectors 205 from image frames. The decoder 160 can be trained to reconstruct the image frames from the feature vectors 205.

In some implementations, the computing system 105 can train models on-premises of a hospital or facility. In some implementation, the models are encoder-decoder models trained with self-supervised training techniques, and no annotations or labels are needed for training. After each on-premises training session, the newly trained decoder can be transmitted to a cloud or server for it to be used to decode images from the new type of feature vectors and the encoder can be retained on-premises. This can allow for models to be customized for specific sites or hospitals, thus improving the reconstruction quality of similar numbers of bits (e.g., or same quality with less bits). In some implementations, a model detecting in or out of body frames can be trained with a similar training techniques.

The machine learning engine 150 can execute one or multiple training algorithms or techniques to train the encoder 130 and the decoder 160 with training data 155 to generate the feature vectors 205 and the reconstructions 615. Responsive to completing training, the machine learning engine 150 can deploy the encoder 130 to the computing system 105 to encode the image frames 125 and deploy the decoder 160 to the remote server 110 to decode feature vectors 205 and reconstruct the image frames 125. The machine learning engine 150 can transmit the encoder 130 to the computing system 105 for implementation. The machine learning engine 150 can transmit the decoder 160 to the remote server 110 for implementation.

The machine learning engine 150 can train the encoder 130 and the decoder 160 with self-supervised machine learning technique for joint embeddings, e.g., self-distillation with no labels (DINO) or masked Siamese network (MSN), auto encoders (AE), decoder-encoder models, or masked auto-encoders (MAEs). The machine learning engine 150 can train the encoder 130 and the feature decoder 160 with a supervised or self-supervised learning method.

The machine learning engine 150 can generate, determine, or calculate losses. For example, the machine learning engine 150 can include a comparator 605 that generates an image loss 625. The comparator 605 can compare images of the training data 155 with reconstructed images 615. The image loss 625 can represent a difference between the images of the training data 155 and the reconstructed images 615. The greater the match between the images, the lower the image loss 625. The lower the match between the images, the greater the image loss 625. The comparator 605 can compare pixels of the original image of the training data 155 and the reconstructed image 615 against each other to determine the loss 625. The comparator 605 can utilize naïve approach, such as calculating a mean squared error to represent the image loss 625. The comparator 605 can be an image comparison model. The comparator 605 can implement mean sum of absolute difference or mean squared error (MSE) to determine loss. The comparator 605 can determine conceptual loss, in which a feature vector extracted from a deep neural network, such as a visual geometry group (VGG) neural network, can be used to compare two images. A VGG neural network can include convolutional layers (e.g., two dimensional convolutional layers) and at least one max pooling layer.

The machine learning engine 150 can include an entropy calculator 610. The entropy calculator 610 can generate an entropy level 620 for the feature vectors 205. The entropy level 620 can represent a level of disorder or how heterogenous the feature vectors 205 are. The higher the level of entropy, the less heterogenous the feature vectors 205 are. The machine learning engine 150 can perform a training or learning algorithm, function, or technique, to decrease or minimize the entropy 620 and decrease or minimize the image loss 625. The machine learning engine 150 can balance the image loss 625 and the entropy 620. Decreasing the entropy 620 and decreasing the image loss 625 can ensure the quality of the reconstructed images 615 are high but the input images 155 are effectively compressed into the feature vectors 205. The smaller the entropy 620, the greater the compression and reduction in data size, but the poorer the image reconstruction. Therefore, the entropy 620 and the image loss 625 can be balanced. The machine learning engine 150 can perform backpropagation, gradient descent, stochastic gradient descent, second order gradient descent, newton method, conjugate gradient, quasi-newton method, or Levenberg-Marquardt algorithm to tune, adjust, or change values of the various parameters and weights of the encoder 130 and decoder 160 to balance the image loss 625 and the entropy 620.

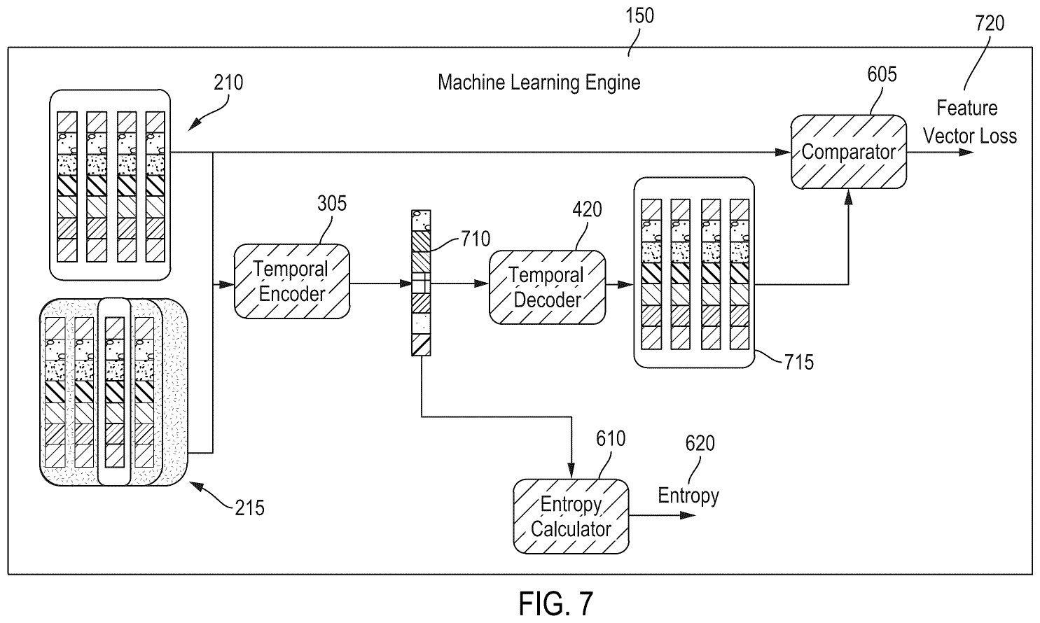

Referring now to FIG. 7, among others, an example computing system 105 to train a temporal encoder 305 to generate a feature vector 710 from a cluster 210 of features 205 and a temporal decoder 420 to generate the cluster 715 from the feature vector 710 is shown. The machine learning engine 150 can train the temporal encoder 305 to generate a feature vector 710 that encodes differences between feature vectors 205 of a cluster 210 of feature vectors 205. The machine learning engine 150 can generate a reconstruction 715 of the cluster 210. The reconstruction 715 can include reconstructed feature vectors that are reconstructions of the feature vectors 205 of the cluster 210.

Responsive to completing training, the machine learning engine 150 can deploy the temporal encoder 305 to the computing system 105 to encode the clusters 210 and deploy the temporal decoder 420 to the remote server 110 to decode feature vectors 710 and reconstruct the clusters 210 of feature vectors 205. The machine learning engine 150 can transmit the temporal encoder 305 to the computing system 105 for implementation. The machine learning engine 150 can transmit the temporal decoder 420 to the remote server 110 for implementation.

The machine learning engine 150 can train the temporal encoder 305 and the temporal decoder 420 with self-supervised machine learning technique for joint embeddings, e.g., self-distillation with no labels (DINO) or masked Siamese network (MSN), auto encoders (AE), decoder-encoder models, masked auto-encoders (MAEs). The machine learning engine 150 can train the temporal encoder 305 and the temporal decoder 420 with a supervised or self-supervised learning method.

The machine learning engine 150 can generate, determine, or calculate losses. For example, the machine learning engine 150 can include a comparator 605 that generates a feature vector loss 720. The comparator 605 can compare clusters 210 of training data 155 with reconstructed clusters 715. The feature vector loss 720 can represent a difference between the clusters. The greater the match between the clusters, the lower the feature vector loss 720. The lower the match between the clusters, the greater the feature vector loss 720. The comparator 605 can compare values of the feature vectors 205 of the original cluster 210 of the training data 155 and the reconstructed feature vectors of the cluster 715 against each other to determine the feature vector loss 720. The comparator 605 can calculating a mean squared error to represent the feature vector loss 720.

The machine learning engine 150 can include an entropy calculator 610. The entropy calculator 610 can generate an entropy level 620 for the feature vectors 710. The entropy level 620 can represent a level of disorder or how heterogenous the feature vectors 710 are. The higher the level of entropy, the less heterogenous the feature vectors 710 are. The machine learning engine 150 can perform a training or learning algorithm, function, or technique, to decrease or minimize the entropy 620 and decrease or minimize the feature vector loss 720. The machine learning engine 150 can balance the feature vector loss 720 and the entropy 620. Decreasing the entropy 620 and decreasing the feature vector loss 720 can ensure the quality of the reconstructed clusters 715 are high and effectively compress the input clusters 210. The machine learning engine 150 can perform backpropagation, gradient descent, stochastic gradient descent, second order gradient descent, newton method, conjugate gradient, quasi-newton method, or Levenberg-Marquardt algorithm) to tune, adjust, or change values of the various parameters and weights of the temporal encoder 305 and temporal decoder 420 to balance the feature vector loss 720 and the entropy 620.

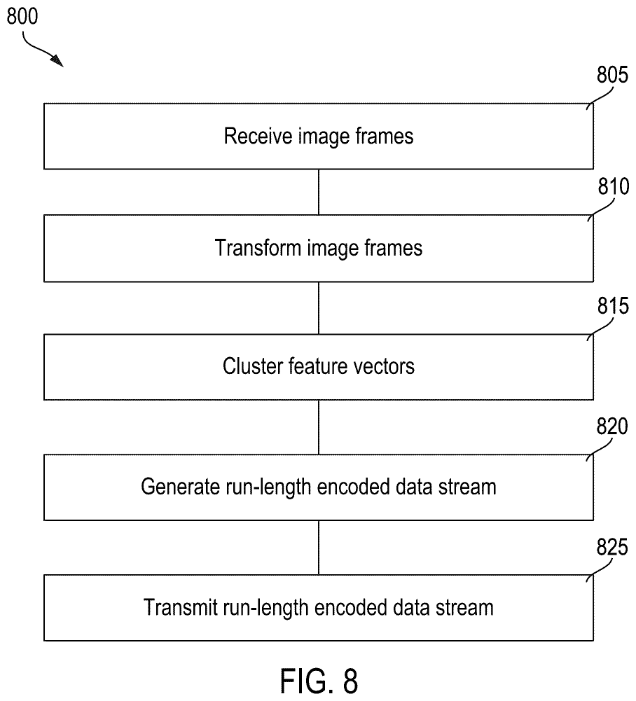

Referring now to FIG. 8, among others, an example method 800 of extracting features 205 of image frames 125 of a medical procedure and generating a run-length encoded data stream 190 from the features 205 is shown. The computing system 105, the robotic medical system 115, the remote server 110, or the client device 170 can perform at least a portion of the method 800. The method 800 can include an ACT 805 of receiving image frames. The method 800 can include an ACT 810 of transforming image frames. The method 800 can include an ACT 815 of clustering feature vectors. The method 800 can include an ACT 820 of generating run-length encoded data stream. The method 800 can include an ACT 825 of transmitting run-length encoded data stream.

At ACT 805, the method 800 can include receiving, by the computing system 105, image frames 125. The method 800 can include capturing, by a camera 120 of the robotic medical system 115, the image frames 125 as the robotic medical system 115 performs a medical procedure. The method 800 can include transmitting, by the robotic medical system 115, the image frames 125 to the computing system 105 intraoperatively (e.g., as the medical procedure is performed) or postoperatively (e.g., after the medical procedure is performed).

At ACT 810, the method 800 can include transforming, by the computing system 105, the image frames 125. The method 800 can include transforming, by the feature extractor 130, the image frames 125 into feature vectors 205. The method 800 can include encoding, by the feature extractor 130, each image frame 125 into a feature vector 205 that represents the information in the original image frames 125 in a compact or compressed manner.

At ACT 815, the method 800 can include clustering, by the computing system 105, the feature vectors 205. The method 800 can include executing, by the clusterer 135, to cluster the feature vectors 205 into different clusters 210. The method 800 can include segmenting a sequence of feature vectors 205 corresponding to the sequence of image frames 125. The method 800 can include segmenting the sequence of feature vectors 205 temporally based on actions or events represented by the feature vectors 205. The method 800 can include selecting a beginning timestamp and an ending timestamp for each cluster 210, e.g., identifying a beginning feature vector 205 and an ending feature vector 205 for each cluster 210.

At ACT 820, the method 800 can include generating, by the computing system 105, a run-length encoded data stream 190. The method 800 can include compressing the information or data of the data stream 190 by reducing consecutive symbols or pieces of information to a single representation and an indication of the number of repetitions of the symbol or piece of information. The method 800 can include, in some implementations, performing a lossless or lossy compression using the clustered features 205 to produce the data stream 190.

At ACT 825, the method 800 can include transmitting, by the computing system 105, the run-length encoded data stream 190 to the remote server 110. The computing system 105 can transmit the run-length data stream 190 to the remote server 110 over the network 175. For example, as new pieces or blocks of data are generated by the run-length encoder 145, the method 800 can include streaming, by the computing system 105, the new pieces of information to the remote server 110. The method 800 can include executing steps to decode the run-length encoded data stream via run-length decoding. The method 800 can include implementing de-scanning to de-scan the data stream 190. The method 800 can include performing inverse quantization on the de-scanned data stream 190. The method 800 can include performing temporal decoding to transform the inverse quantized data stream 190 into the cluster 210 of feature vectors 205. The method 800 can include transforming the feature vectors 205 of the clusters 210 back into the image frames 125 using a feature decoder 160. The feature decoder 160 can be trained with the encoder of the feature extractor 130, e.g., the feature decoder 160 and the feature extractor 130 can be paired together.

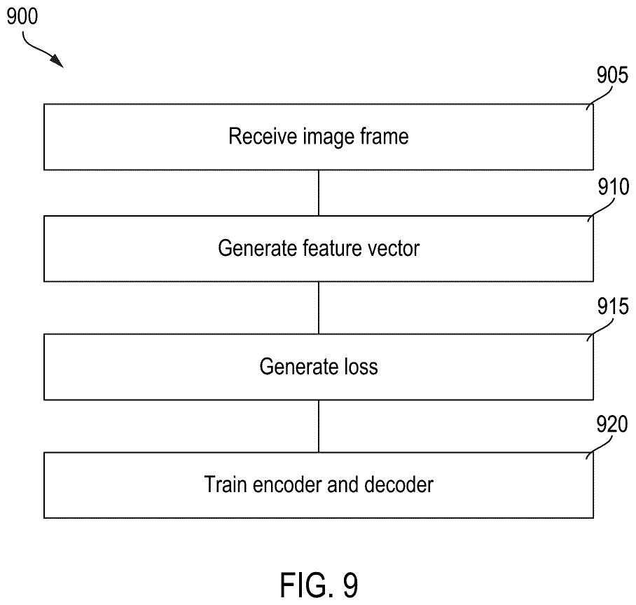

Referring now to FIG. 9, among others, an example method 900 of training an encoder 130 to extract features 205 from image frames 125 and a decoder 180 to reconstruct the image frames 125 from the extracted features 205 is shown. The computing system 105, the robotic medical system 115, the remote server 110, or the client device 170 can perform at least a portion of the method 900. The method 900 can include an ACT 905 of receiving an image. The method 900 can include an ACT 910 of generating a feature vector. The method 900 can include an ACT 915 of generating a loss. The method 900 can include an ACT 920 of training an encoder and decoder. While FIG. 9 is described with respect to training the encoder 130 and the decoder 180, the techniques described with reference to FIG. 9 can be performed as a method to train the temporal encoder 305 and the temporal decoder 420.

At ACT 905, the method 900 can include receiving, by the machine learning engine 150, an image frame. For example, the computing system 105 can receive training data 155 that can include multiple image frames. The method 900 can include filtering the privacy filter 185 to remove images from the training data 155 that include private information. The method 900 can include generating the training data 155 iteratively or continuously as new image frames 125 are received form the robotic medical system 115. For example, the method 900 can include updating or expanding the training data 155 as image frames 125 are received from the robotic medical system 115 as the robotic medical system 115. Responsive to a predefined number of image frames 125 being added to the training data 155, the method 900 can include triggering or initiating training or re-training of the feature extractor 130 and the feature decoder 160.

At ACT 910, the method 900 can include generating, by the machine learning engine 150, a feature vector 205. The method 900 can include executing, by the machine learning engine 150 an encoder 130. The method 900 can include executing the encoder 130 to transform the image 155 from a first space to a second feature space. The method 900 can include executing the encoder 130 to generate a set of values, numbers, or labels in a vector that represent various features which describe the image frame 155.

At ACT 915, the method 900 can include generating, by the machine learning engine 150, a loss. The method 900 can include executing a decoder 160 to generate, reproduce, or reconstruct the original image 155. The method 900 can include executing the decoder 160 to generate the image frames 615 from the feature vectors 205. The method 900 can include transforming the feature vectors 205 from a feature space back into an image space. The method 900 can include generating, by the machine learning engine 150, an image loss 625. The method 900 can include executing a comparator 605 to compare an original image 155 with a reconstructed image 615 to produce the image loss 625. The image loss 625 can measure a level of how closely the original image 155 and the reconstructed image 615 are. The image loss 625 can be a mean squared error between the original image 155 and the reconstructed image 615. The method 900 can include generating, by the machine learning engine 150, an entropy 620. The method 900 can include executing an entropy calculator 610 to output an entropy 620 that measures a level of entropy within a set of feature vectors 205. The level of entropy can indicate how heterogenous the feature vectors 205 are.

At ACT 920, the method 900 can include training, by the machine learning engine 150, the encoder 130 and the decoder 160. The method 900 can include executing, by the machine learning engine 150, a technique, algorithm, or process that minimizes or maximizes losses. The method 900 can include executing, by the machine learning engine 150, a technique, algorithm, or process that balances losses, for example, minimize one loss and maximizes another loss. The method 900 can include adjusting, changing, or setting parameters, weights, or values of the encoder 130 and the decoder 160 to decrease the entropy 620 and decrease the image loss 625. The method 900 can include performing, by the machine learning engine 150, backpropagation, gradient descent, stochastic gradient descent, second order gradient descent, newton method, conjugate gradient, quasi-newton method, or Levenberg-Marquardt algorithm to tune, adjust, or change values of the various parameters and weights of the encoder 130 and decoder 160 to balance the image loss 625 and the entropy 620.

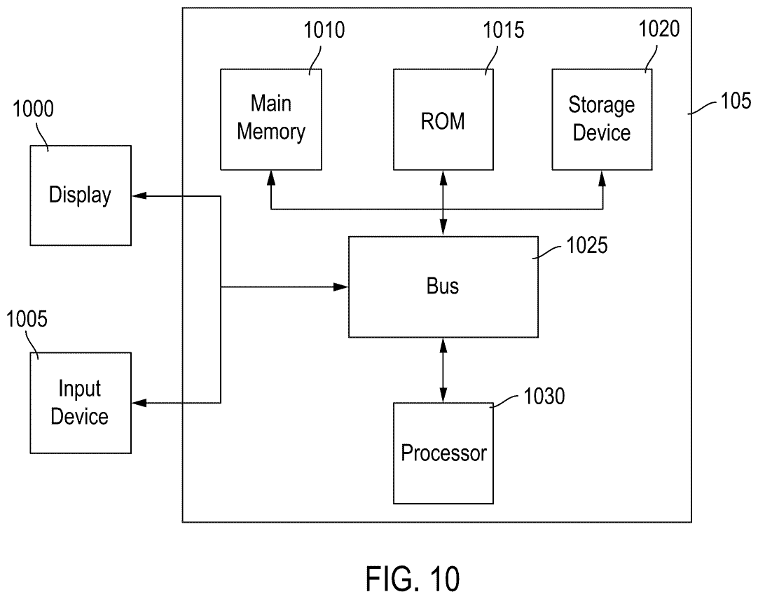

Referring now to FIG. 10, among others, an example block diagram of a computing system 105 is shown. The computing system 105 can include or be used to implement a data processing system or its components. The architecture described in FIG. 10 can be used to implement the computing system 105, the robotic medical system 115, or the remote server 110. The computing system 105 can include at least one bus 1025 or other communication component for communicating information and at least one processor 1030 or processing circuit coupled to the bus 1025 for processing information. The computing system 105 can include one or more processors 1030 or processing circuits coupled to the bus 1025 for processing information. The computing system 105 can include at least one main memory 1010, such as a random access memory (RAM) or other dynamic storage device, coupled to the bus 1025 for storing information, and instructions to be executed by the processor 1030. The main memory 1010 can be used for storing information during execution of instructions by the processor 1030. The computing system 105 can further include at least one read only memory (ROM) 1015 or other static storage device coupled to the bus 1025 for storing static information and instructions for the processor 1030. A storage device 1020, such as a solid state device, magnetic disk or optical disk, can be coupled to the bus 1025 to persistently store information and instructions.

The computing system 105 can be coupled via the bus 1025 to a display 1000, such as a liquid crystal display, or active matrix display. The display 1000 can display information to a user. An input device 1005, such as a keyboard or voice interface can be coupled to the bus 1025 for communicating information and commands to the processor 1030. The input device 1005 can include a touch screen of the display 1000. The input device 1005 can include a cursor control, such as a mouse, a trackball, or cursor direction keys, for communicating direction information and command selections to the processor 1030 and for controlling cursor movement on the display 1000.

The processes, systems and methods described herein can be implemented by the computing system 105 in response to the processor 1030 executing an arrangement of instructions contained in main memory 1010. Such instructions can be read into main memory 1010 from another computer-readable medium, such as the storage device 1020. Execution of the arrangement of instructions contained in main memory 1010 causes the computing system 105 to perform the illustrative processes described herein. One or more processors in a multi-processing arrangement can be employed to execute the instructions contained in main memory 1010. Hard-wired circuitry can be used in place of or in combination with software instructions together with the systems and methods described herein. Systems and methods described herein are not limited to any specific combination of hardware circuitry and software.

Although an example computing system has been described in FIG. 10, the subject matter including the operations described in this specification can be implemented in other types of digital electronic circuitry, or in computer software, firmware, or hardware, including the structures disclosed in this specification and their structural equivalents, or in combinations of one or more of them.

Some of the description herein emphasizes the structural independence of the aspects of the system components or groupings of operations and responsibilities of these system components. Other groupings that execute similar overall operations are within the scope of the present application. Modules can be implemented in hardware or as computer instructions on a non-transient computer readable storage medium, and modules can be distributed across various hardware or computer based components.

The systems described above can provide multiple ones of any or each of those components and these components can be provided on either a standalone system or on multiple instantiations in a distributed system. In addition, the systems and methods described above can be provided as one or more computer-readable programs or executable instructions embodied on or in one or more articles of manufacture. The article of manufacture can be cloud storage, a hard disk, a CD-ROM, a flash memory card, a PROM, a RAM, a ROM, or a magnetic tape. In general, the computer-readable programs can be implemented in any programming language, such as LISP, PERL, C, C++, C#, PROLOG, Python, or in any byte code language such as JAVA. The software programs or executable instructions can be stored on or in one or more articles of manufacture as object code.

Example and non-limiting module implementation elements include sensors providing any value determined herein, sensors providing any value that is a precursor to a value determined herein, datalink or network hardware including communication chips, oscillating crystals, communication links, cables, twisted pair wiring, coaxial wiring, shielded wiring, transmitters, receivers, or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), or digital control elements.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices including cloud storage). The operations described in this specification can be implemented as operations performed by a data processing apparatus on data stored on one or more computer-readable storage devices or received from other sources.

The terms “computing device”, “component” or “data processing apparatus” or the like encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data can include non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. ACTs, elements and features discussed in connection with one implementation are not intended to be excluded from a similar role in other implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any ACT or element being based on any information, act or element may include implementations where the act or element is based at least in part on any information, act, or element.

Any implementation disclosed herein may be combined with any other implementation or example, and references to “an implementation,” “some implementations,” “one implementation” or the like are not necessarily mutually exclusive and are intended to indicate that a particular feature, structure, or characteristic described in connection with the implementation may be included in at least one implementation or example. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

What is claimed is:

1. A system, comprising:
one or more processors, coupled with memory, to:
receive, via a robotic medical system, a plurality of image frames related to a medical procedure performed by the robotic medical system;
transform, via one or more models trained with machine learning on historical images of medical procedures, the plurality of image frames to a plurality of feature vectors;
cluster, via the one or more models, the plurality of feature vectors into a plurality of clusters;
generate a run-length encoded data stream based at least in part on the plurality of clusters; and
transmit, via a network, the run-length encoded data stream to one or more servers remote from the one or more processors to manage performance of the medical procedure.

2. The system of claim 1, comprising the one or more processors to:
train, using machine learning, the one or more models to reduce image loss between the plurality of image frames and the plurality of feature vectors.

3. The system of claim 1, comprising the one or more processors to:
train, using machine learning, the one or more models to decrease entropy in the plurality of feature vectors.

4. The system of claim 1, comprising the one or more processors to:
execute a function to train the one or more models that reduces image loss and decreases entropy in the plurality of feature vectors.

5. The system of claim 1, comprising the one or more processors to:
receive a data set comprising the historical images;
filter the historical images to remove first images comprising private medical data from the data set and retain second images comprising non-private medical data; and
train, using machine learning, the one or more models with the filtered historical images to generate first feature vectors for the first images, and generate second feature vectors for the second images, wherein images reconstructed from the second feature vectors have a level of accuracy that is less than images reconstructed from the first feature vectors.

6. The system of claim 1, wherein a cluster of the plurality of clusters includes at least two of the plurality of feature vectors.

7. The system of claim 1, comprising the one or more processors to:
train, using machine learning, an encoder to generate a feature vector from an image frame and a decoder to generate the image frame from the feature vector; and
deploy the decoder to the one or more servers to execute on the one or more servers to transform the feature vector into the image frame responsive to the feature vector being extracted from the run-length encoded data stream.

8. The system of claim 1, comprising the one or more processors to:
select a representative feature vector from a plurality of feature vectors for a cluster of the plurality of clusters; and
generate, using the representative feature vector, run-length encoded data to represent the plurality of feature vectors of the cluster.

9. The system of claim 1, comprising the one or more processors to:
receive the run-length encoded data stream comprising run-length encoded data generated to represent a plurality of feature vectors of a cluster;
generate, using the run-length encoded data stream, the plurality of feature vectors of the cluster; and
decode, using one or more second models, the plurality of feature vectors of the cluster into at least a portion of the plurality of image frames.

10. The system of claim 1, comprising the one or more processors to:
receive the run-length encoded data stream comprising run-length encoded data generated to represent a plurality of feature vectors of a cluster;
generate, using the run-length encoded data, the plurality of feature vectors of the cluster; and
classify, using one or more second models and the plurality of feature vectors of the cluster, each feature vector of the plurality of feature vectors of the cluster into a class of a plurality of classes.

11. The system of claim 1, comprising the one or more processors to:
receive the run-length encoded data stream comprising run-length encoded data generated to represent a plurality of feature vectors of a cluster;
generate, using the run-length encoded data, the plurality of feature vectors of the cluster; and
label, using one or more second models and the plurality of feature vectors of the cluster, an action performed by the robotic medical system represented in each feature vector of the plurality of feature vectors of the cluster.

12. The system of claim 1, comprising the one or more processors to:
receive an indication of the medical procedure;
select, using the indication of the medical procedure, a bit rate;
quantize, using the bit rate, the plurality of clusters; and
generate the run-length encoded data stream from the quantized plurality of clusters.

13. A method, comprising:
receiving, by one or more processors, coupled with memory, via a robotic medical system, a plurality of image frames related to a medical procedure performed by the robotic medical system;
generating, by the one or more processors, via one or more models trained with machine learning on historical images of medical procedures, a plurality of feature vectors based on the plurality of image frames;
clustering, by the one or more processors, via the one or more models, the plurality of feature vectors into a plurality of clusters;
generating, by the one or more processors, a run-length encoded data stream based at least in part on the plurality of clusters; and
transmitting, by the one or more processors, via a network, the run-length encoded data stream to one or more servers remote from the one or more processors to manage performance of the medical procedure.

14. The method of claim 13, comprising:
training, by the one or more processors, using machine learning, the one or more models to reduce image loss between the plurality of image frames and the plurality of feature vectors.

15. The method of claim 13, comprising:
executing, by the one or more processors, a function to train the one or more models that reduces image loss and decreases entropy in the plurality of feature vectors.

16. The method of claim 13, comprising:
receiving, by the one or more processors, a data set comprising the historical images;
filtering, by the one or more processors, the historical images to remove first images comprising private medical data from the data set and retain second images comprising non-private medical data; and
training, by the one or more processors, using machine learning, the one or more models with the filtered historical images to generate first feature vectors for the first images, and generate second feature vectors for the second images, wherein images reconstructed from the second feature vectors have a level of accuracy that is less than images reconstructed from the first feature vectors.

17. The method of claim 13, comprising:
training, by the one or more processors, using machine learning, an encoder to generate a feature vector from an image frame and a decoder to generate the image frame from the feature vector; and
deploying, by the one or more processors, the decoder to the one or more servers to execute on the one or more servers to transform the feature vector into the image frame responsive to the feature vector being extracted from the run-length encoded data stream.

18. The method of claim 13, comprising;
selecting, by the one or more processors, a representative feature vector from a plurality of feature vectors for a cluster of the plurality of clusters; and
generating, by the one or more processors, using the representative feature vector, run-length encoded data to represent the plurality of feature vectors of the cluster.

19. A non-transitory computer-readable medium storing processor-executable instructions that, when executed by one or more processors, cause the one or more processors to:
receive, via a robotic medical system, a plurality of image frames related to a medical procedure performed by the robotic medical system;
generate, via one or more models trained with machine learning on historical images of medical procedures, a plurality of clusters of a plurality of feature vectors based on the plurality of image frames;
construct a run-length encoded data stream based at least in part on the plurality of clusters; and
transmit, via a network, the run-length encoded data stream to one or more servers remote from the one or more processors to manage performance of the medical procedure.

20. The non-transitory computer-readable medium of claim 19, wherein the instructions cause the one or more processors to:
receive a data set comprising the historical images;
filter the historical images to remove first images comprising private medical data from the data set and retain second images comprising non-private medical data; and
train, using machine learning, the one or more models with the filtered historical images to generate first feature vectors for the first images, and generate second feature vectors for the second images, wherein images reconstructed from the second feature vectors have a level of accuracy that is less than images reconstructed from the first feature vectors.
